(19) United States
(12) Patent Application Publication
(10) Pub. No.: US 2011/0237929 A1
(43) Pub. Date: Sep. 29, 2011

(54) BLADDER WALL THICKNESS MAPPING FOR TUMOR DETECTION

(76) Inventors: Zhengrong Liang, Stony Brook, NY (US); Su Wang, San Jose, CA (US); Chaijie Duan, Port Jefferson, NY (US); Hongbin Zhu, Centereach, NY (US); Xianfeng Gu, Plainview, NY (US)

(21) Appl. No.: 13/062,649

(22) PCT Filed: Sep. 8, 2009
(86) PCT No.: PCT/US09/56248

§ 371 Date: May 31, 2011

Related U.S. Application Data
(60) Provisional application No. 61/094,463, filed on Sep. 5, 2008.
(60) Provisional application No. 61/239,862, filed on Sep. 4, 2009.

(51) Int. Cl. A61B 5/055

(52) U.S. Cl. 600/410

(57) ABSTRACT

Disclosed is a method and apparatus for detection of a bladder wall tumor. Layers of a bladder wall are created by magnetic resonance imaging. A group of voxels having a lowest intensity is identified in a layer and an energy function modification enlarges the layer of the bladder wall. A partial volume image segmentation obtains tissue type mixture percentages in each voxel near inner and outer borders of the bladder wall in the layer of the bladder wall to obtain a bladder wall thickness. A range of uncertainty at the inner and outer borders of the bladder wall is obtained, and integration is performed of the bladder wall thickness along a path starting at a point on the outer border and ending at a corresponding point on the inner border.

### PRIORITY

This application claims priority to U.S. Provisional Application No. 61/094,463, filed Sep. 5, 2008, and to U.S. Provisional Application No. 61/239,862, filed Sep. 4, 2009, the contents of each of which is incorporated herein by reference.

### BACKGROUND OF THE INVENTION

1. Field of the Invention

The present invention relates to detection of bladder tumors via magnetic resonance imaging and computer-aided detection.

2. Brief Description of the Background Art

Bladder carcinoma is becoming the fifth leading cause of cancer-related deaths in the United States, occurring in a 3:1 ratio between men and women. The lifetime probability of developing bladder carcinoma is over three percent, and the probability of dying from this cancer is approximately one percent, a thirty-five percent increase over the past decade. It was estimated that 50,000 new cases and 11,000 deaths occurred in 1995, increasing to 68,500 new cases and 14,000 deaths in 2008. Approximately 75% of the cancers were reported as a localized disease, 19% were reported as regional metastases, 3% were reported as distant metastases, and remaining were reported as other metastases. Pathologically, more than 90% of bladder cancer cases occur as pure transitional cell carcinoma. The remaining less than 10% of bladder cancer cases are divided between squamous cell carcinomas (5% to 7%), adenocarcinomas (1% to 2%), and sarcomas (1% to 2%). Approximately 70% of the transitional cell carcinomas are superficial or papillary tumors. The remaining 30% are invasive.

Moreover, bladder cancer has very high recurrence rate after resection, with a recurrence rate as high as 80%. Therefore, early diagnosis of bladder abnormalities is crucial for effective treatment of bladder carcinoma. As a main method of investigating bladder abnormalities, fiber optic cystoscopy (OCys) is accurate and can perform a biopsy when a tumor is found. However, OCys is invasive, time-consuming, expensive, uncomfortable, incapable of viewing the entire bladder mucosa, and has the risk of urinary tract infection.

Early asymptomatic bladder cancer may be associated with occult bleeding (microscopic hematuria) or the presence of dysplastic cells in the urine. Urine dipsticks or standard urinalysis, which detect peroxidase activity of hemoglobin and can be performed at home, provide a quick, safe, and inexpensive test for hematuria with a high sensitivity of approximately 90%. However, such conventional testing has a very poor specificity, as low as 65%, because other causes can lead to microscopic hematuria, such as benign prostatic hypertrophy (BPH), exercise, renal cysts, urethral trauma, menstrual bleeding, bladder stones, dysplasia, and asymptomatic infection. Furthermore, such conventional testing cannot provide accurate location and information on the tumor staging. Evaluation of asymptomatic microscopic hematuria is very complicated and costly.

Other highly sensitive methods for detection of high-grade urothelial malignancy, in addition to the urine dipsticks, include urine cytology, Fluorescence In Situ Hybridization (FISH), and Bladder Tumor Antigen. However, these methods share the same limitations as urine dipsticks in providing the location and staging of the tumor.

Among the minimal or non-invasive tests with accurate location, such as ultrasound, X-ray angiography and Computed Tomography (CT), Intravenous Pyelography (IVP) is the standard radiological test used in the evaluation of a patient with hematuria. However, IVP carries the risk of allergic reaction, nephrotoxicity, and radiation exposure. Despite its utility, IVP does not demonstrate small bladder tumors, and fiber optic cystoscopy must be performed to evaluate the urinary bladder.

Fiber optic cystoscopy, a mandatory part of the evaluation of a patient with hematuria for bladder abnormalities, is more accurate, because most tumors (more than 90%) appear as small growths rising from the inner surface of the bladder wall in forms of polypoid, sessile, or abnormal plaques. This method is performed, when the patient is placed in a lithotomy position, by inserting an endoscope through the urethra into the bladder. The method was reported with a sensitivity of approximately 87% and specificity of approximately 95%. However, it is invasive, time-consuming, expensive, and uncomfortable, with a risk of 5% to 10% rate of urinary tract infection (and a higher rate of bleeding) following the invasive procedure. Due to the low specificity of standard urinalysis/IVP and the difficulty of fiber optic cystoscopy, the finding of bladder cancer is usually at a very late stage, resulting in a high morbidity and mortality, as well as a high cost of patient management.

Recently, CT-based and Magnetic Resonance (MR) based virtual cystoscopy (VCys) have been developed as an alternative means for bladder cancer detection and evaluation. Such methods are safe, less or non-invasive, and less expensive as compared to OCys. In CT or MR bladder images, it is expected that early signs of bladder lesion would be reflected by both the morphology and texture on the bladder wall and mucosa. However, radiologists must read the image slices one-by-one to locate possible abnormalities. Accordingly, three-dimensional endoscopic views on the mucosa can be made available to assist the detection. However, such a reading process is time-consuming and brings fatigue error of diagnosis.

Fortunately, Computer Aided Detection (CAD) of bladder tumors has shown potential to be a second reader to help radiologists improve their performance. At early stages, flat and/or small tumors of less than 5 mm are difficult to detect and, therefore, deserve more attention. Most bladder cancers originate in the epithelial cells, e.g. the uroepithelial cells, and are treatable if diagnosed prior to metastasis and if managed appropriately. Therefore, early detection is crucial to prevent the disease and reduce the death rate.

Conventional characteristic features on the bladder wall, like curvedness and shape index, vary significantly from voxel to voxel. In contrast, for a small bump protruding out of the bladder wall, the measurement of the thickness between the inner and outer borders tends to be a good indicator of the occurrence of abnormalities. See, U.S. Pat. No. 7,260,250, the contents of which are incorporated herein by reference.

Conventional VCys are typically based on CT technology, due to high spatial resolution, fast acquisition speed, and wide availability. However, the sensitivity of CT imaging to soft tissues, including urine, prohibits itself from providing good contrast in the bladder wall. This limitation is partly mitigated by the injection of a contrast medium, such as by tagging urine by intravenous injection or emptying the bladder and then filling the bladder with air via a catheter. Unfortunately, not only is this procedure invasive and uncomfortable, but also the CT imaging delivers excessive X-ray exposure to the patients, both of which considerably decrease the patients' compliance.

To avoid these obstacles, MR imaging is a preferred alternative, considering the structural, functional and pathological information for diagnosing and staging the tumor growth. In addition, MR imaging uses endogenous rather than exogenetic contrast medium to alter the image intensity of the bladder wall against its surroundings (urine inside and fat outside) towards a fully non-invasive procedure. Since hydrogen in water (or urine) has longer transverse relaxation time leading to higher intensity values in the T₂-weighted MR images, many previous Magnetic Resonance Image (MRI)-based VCys or MR cystography researchers focused on T₂-weighted imaging, where urine is used as an endogenous contrast medium to enhance the contrast between bladder lumen and wall. In the present invention a method is provided using MR cystography for bladder evaluation.

### SUMMARY OF THE INVENTION

The present invention introduces (1) a method to extract the inner and outer borders; (2) a method to determine the thickness between the inner and outer borders; (3) a method to compute the integrated density or line integral along a path, where the path gives the thickness measured and the line integral provides various features for visualization and CAD; (4) a method to map the thickness distribution/integrated density distribution for visualization; and (5) a CAD scheme for detection of bladder tumors based on the features of the thickness mapping/integrated density mapping of the bladder wall in MR images.

The present invention also provides a non-invasive method for detection of bladder tumors that reduces interpretation time, thereby reducing radiologists fatigue when reading (1) flattened thickness distribution and (2) CAD scheme thickness mapping detection techniques of bladder walls to detect a locally thickened bladder wall, which often appears around tumors.

The present invention uses an MRI cystography system to detect the bladder wall from T₁-weighted and T₂-weighted volume images of the bladder, analyzes the image texture of the extracted wall, and detects the patches where abnormalities are highly likely present for reviewers' assessment, preferably via CAD.

In addition to the use of T₂-weighted images, the present invention uses T₁-weighted images, where the urine image intensity is decreased, instead of increasing in T₂-weighted images. The reason of using T₁-weighted images is two-fold. On one hand, the decreased image intensities of urine in T₁-weighted images provide good contrast between the bladder wall and the bladder lumen. On the other hand, the Partial Volume (PV) effect in T₁-weighted images goes from the wall toward the lumen and, therefore, is less visible, while in T₂-weighted image, the PV goes from the lumen into the wall region and can ‘swallow’ small abnormal growths on the inner border.

### BRIEF DESCRIPTION OF THE DRAWINGS

The above and other objects, features and advantages of certain exemplary embodiments of the present invention will be more apparent from the following detailed description taken in conjunction with the accompanying drawings, in which:

FIG. 1 shows dual MR scans of a patient's bladder according to an embodiment of the present invention;

FIG. 2(a) through 2(d) show image segmentation of the method of the present invention;

FIG. 3(a) provides a two-dimensional presentation of the embodiment of FIG. 1;

FIG. 3(b) further provides two-dimensional presentation of the embodiment of FIG. 1 in a single earth map;

FIG. 4 shows regions of interest according to the present invention;

FIG. 5 shows results from a patient's scans performed in accordance with the present invention;

FIGS. 6(a)-(c) show weighted MR images of the bladder;

FIG. 7 is a flowchart of a method of an embodiment of the present invention;

FIGS. 8(a)-(d) show paths used to measure a thickness between inner and outer borders; and

FIG. 9 is a flowchart of a method of a preferred embodiment of the invention.

### DETAILED DESCRIPTION OF THE PREFERRED EMBODIMENTS

The following detailed description of preferred embodiments of the invention will be made in reference to the accompanying drawings. In describing the invention, an explanation about related functions or constructions known in the art are omitted for the sake of clearness in understanding the concept of the invention, and to avoid obscuring the invention with unnecessary detail.

Bladder carcinoma invades gradually from the mucosa into wall muscles. Depending upon degree of penetration, bladder carcinoma is categorized into different stages. The transition at different stages can be reflected by image geometry and intensity features in the bladder wall. In the present application, the term ‘bladder wall’ is used to indicate a volumetric shell encompassed by inner and outer borders. Geometrical analysis on the wall is a primary tool, as set out herein, with some additional available intensity texture information, for locating bladder lesions by some irregular shape and contrast patterns at a late stage.

To minimize a Partial Volume Effect (PVE) between urine and the bladder wall, T₁ weighted images are acquired as the primary information for detection purposes, where the urine signal is suppressed and the PVE goes from the wall into the lumen, and has less impact on the wall as compared to T₂ weighted images where the urine signal is enhanced and the PVE goes from the lumen into the wall, and would bury small pathological changes on the mucosa.

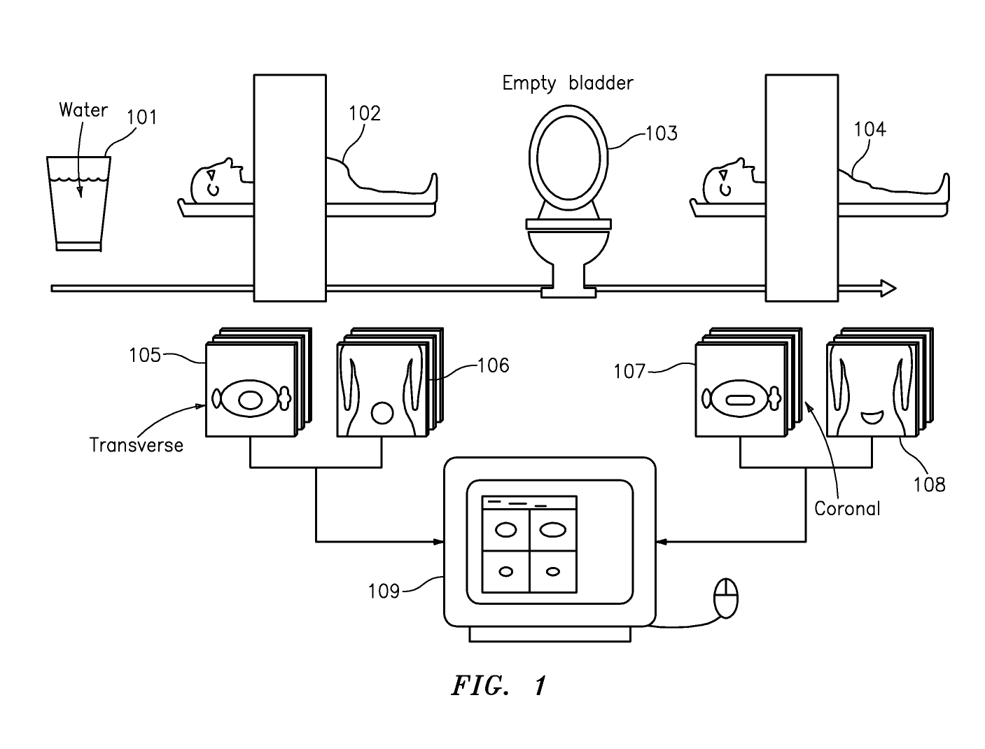

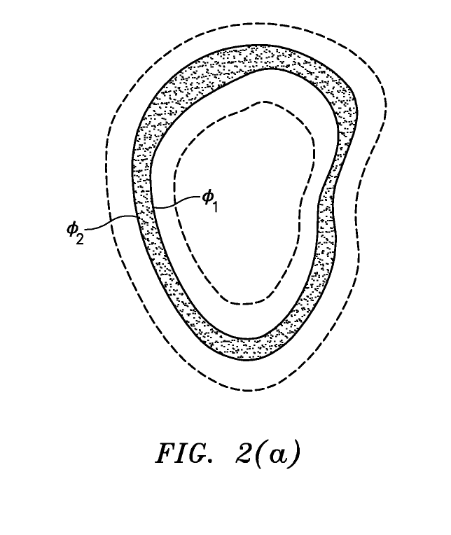

As shown in FIG. 1, a protocol of dual MR scans of the bladder is provided. Preferably, two T₁ weighted scans are acquired after the patient voids the bladder and takes a cup of water. In the middle and final stages (104 and 102 of FIG. 1), a patient has a half-filled bladder and has a fully filled bladder, respectively. Each of the scans of the bladder consists of numerous two-dimensional slice images, stacked together to create a volumetric image, along with transverse images (105, 107) and coronal images (106, 108). A display panel (109) shows the extracted bladder regions from the transverse and coronal images.

The T₁-weighted images were acquired by a whole-body scanner with a body coil transceiver, such as a Philips 1.5T Edge scanner. In this embodiment, an image acquisition protocol includes a 3DFFE-SPIR CLEAR pulse sequence, a 1.5 mm slice thickness, a 10° flip angle, a 448×448 image size with T_{R}=4.6666 ms and a T_{E}=2.2766 ms. Each T₁ volume image is segmented by a hybrid method to search an initial inner border of the bladder by level-set strategy starting from a group of voxels with lowest intensity in the image. The starting point may be given by a T₂-weighted image, such that the initialization can be in an automated manner. From the initial inner border, an enlarged version, or an initial outer border, is obtained by a same level-set strategy with a different energy function

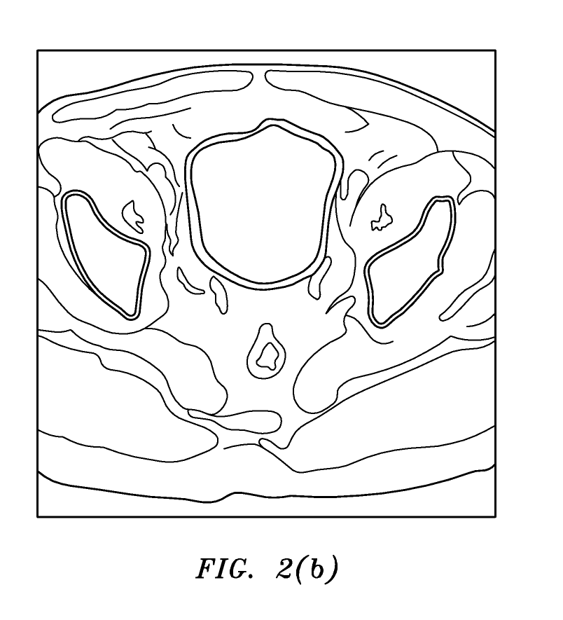

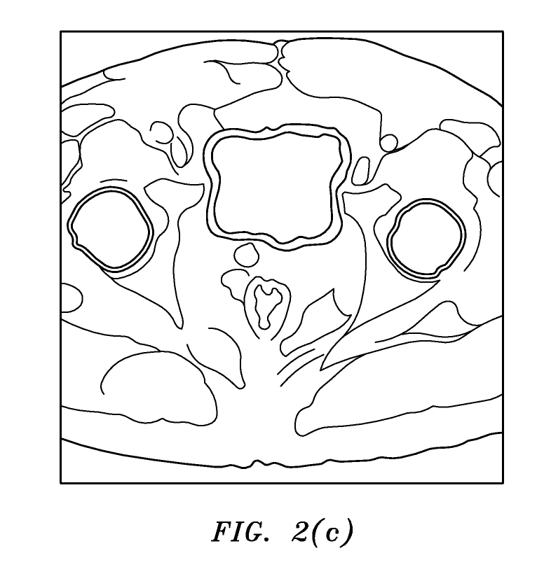

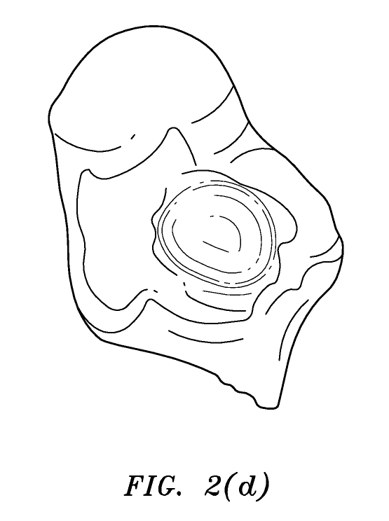

FIG. 2 shows image segmentation of the method of the present invention, with FIG. 2(a) providing a two-dimensional presentation showing dilation of an obtained wall thickness for a sufficiently large layer that includes a PVE on both borders or sides of the wall, further quantified by a PV segmentation algorithm. Each voxel inside the dilated layer after the PV segmentation contains a percentage of three tissue types: urine, wall and fat/muscle mixture outside the wall. Voxels having wall percentages of less than 5% are ignored and the remaining voxels are determined to represent the bladder wall. FIGS. 2(b) and 2(c) provide two examples of dilated layers, and FIG. 2(d) provides an example of an extracted bladder.

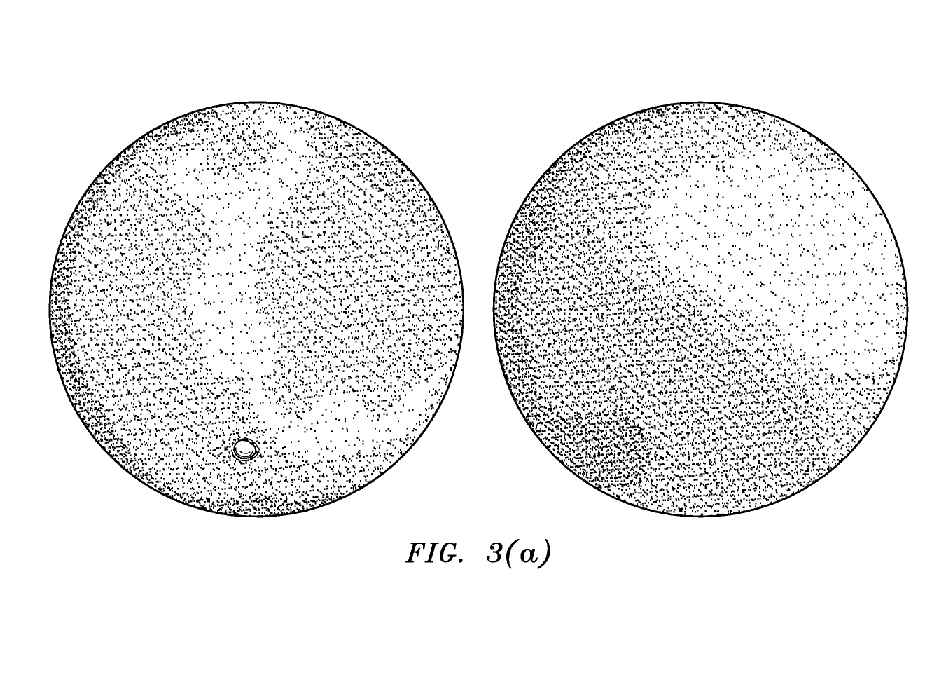

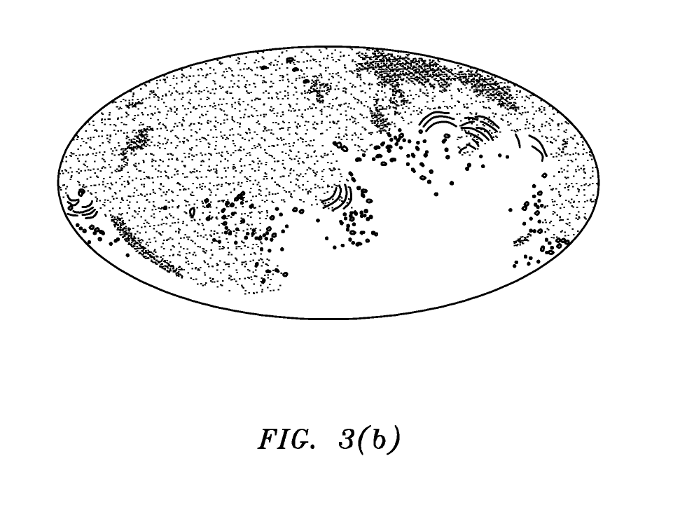

To facilitate clinical use, a conformal flattening strategy is applied to deform the three-dimension object into two-dimensional pictures, in which the three-dimension object is deformed into a sphere, with the deformation on the inner surface of the bladder. The sphere is then flattened into two disks, each representing a half of the sphere. FIG. 3(a) shows an example of two disks flattened from a sphere deformed from a patient's bladder, with wall thickness distribution on the inner surface of the bladder shown in grey scale. Two abnormalities are seen from the left picture in FIG. 3(a). The mapping from the sphere to an earth map is shown in FIG. 3(b).

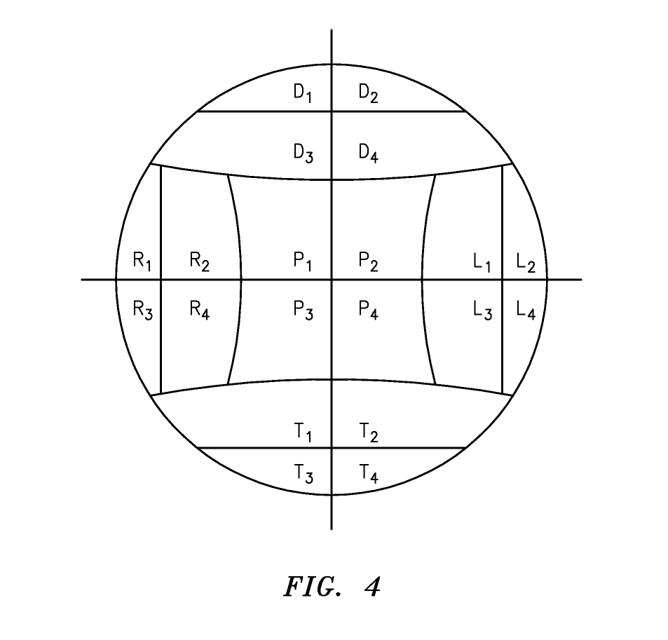

Further specifics of bladder wall details are obtained on the flattening of two disks of FIG. 3(a) or the flattened earth map of FIG. 3(b) by dividing the inner surface of the three-dimensional bladder into Regions Of Interest (ROIs). FIG. 4 is a diagram showing a distribution of the ROIs on a disk from a posterior half sphere of the three-dimensional object. The area around the posterior direction is divided into four ROIs, Pᵢ. The left side and right side are also divided into four ROIs respectively, Lᵢ and Rᵢ. The top dome of the bladder is divided into another four ROIs, Dᵢ. Similarly, the bottom dome is also divided into four ROIs, Tᵢ. The area around the anterior direction is also divided into four ROIs, Bᵢ, similar to the ROIs of Pᵢ (for simplicity, Bᵢ is not shown in FIG. 4). By adding the four Bᵢ's to both sides of FIG. 4, the ROIs on the whole three-dimensional bladder are shown on an earth map of FIG. 3B. Such ROI distribution provides a spatial reference of each detected abnormality on the three-dimensional inner surface of the bladder with correlation to the report of optical cystoscopy, and further provides an image-based guidance for optical cystoscopy intervention to conform and resect the tumor detected by MRI cystography.

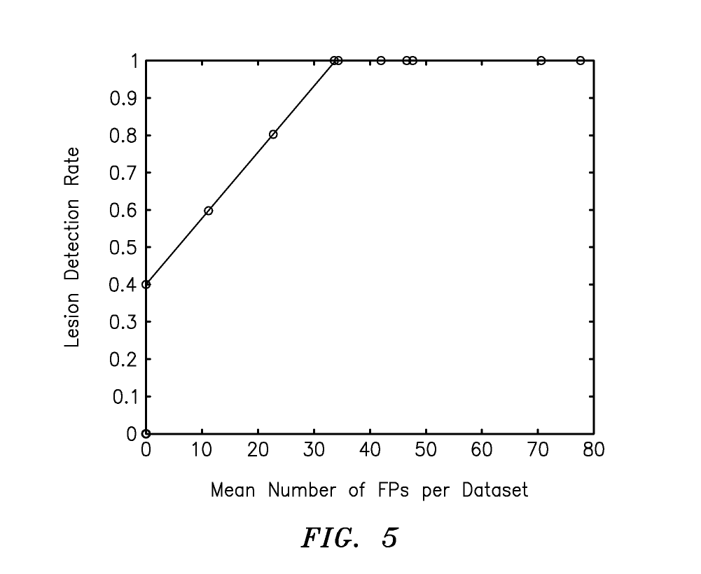

The MRI cystography system was tested on ten MR patient bladder scans with two tumors greater than 10 mm, one of 4 mm, and two less than 3 mm. A Free Response receiver Operating Characteristic (FROC) curve for the automatic CAD of the tumors is shown in FIG. 5. Detection sensitivity reaching 100% with less than thirty-five false positives per patient scan was obtained.

Although early detection of bladder cancer, particularly for tumors of less than 3 mm, remains a challenging task by current clinical MRI scanners with 1.5 mm voxel resolution, the MRI-virtual cystoscopy system of the present invention has demonstrated the potential for evaluation of tumor recurrence that otherwise require patient follow-up with fiber optic cystoscopy every three to six months after tumor resection.

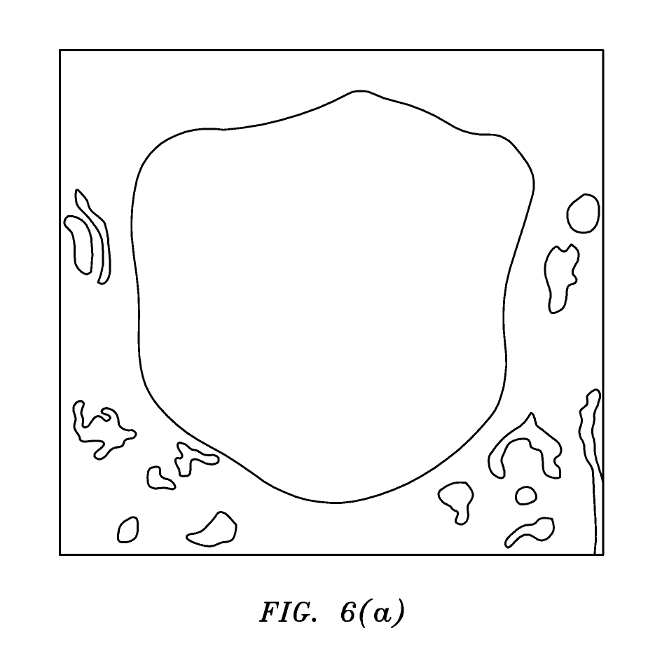

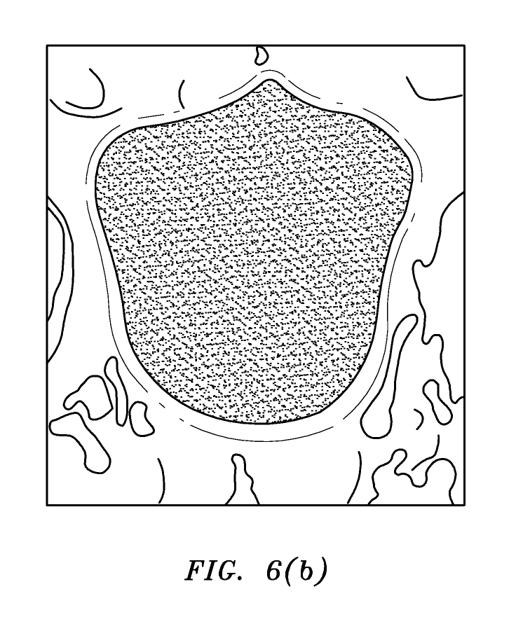

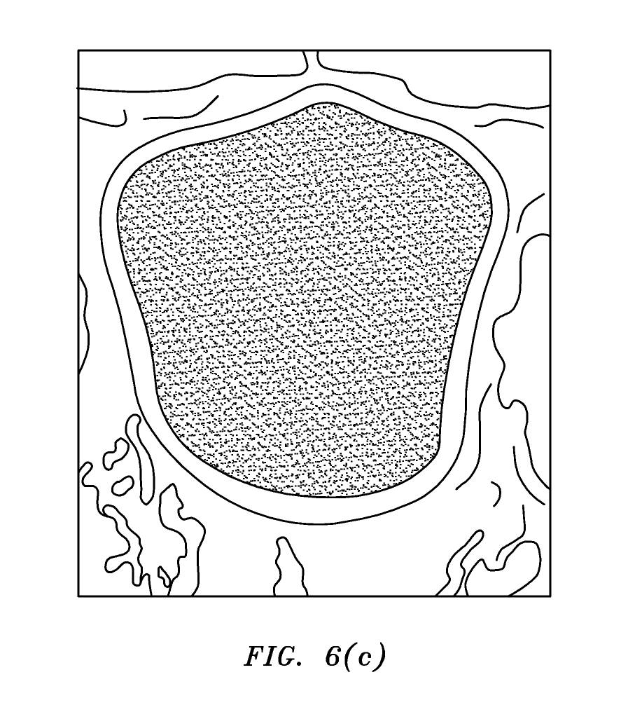

As an overview of the CAD scheme, opposed to T₂-weighted MR images shown in FIG. 6(a), T₁-weighted MR images, as shown in FIG. 6(b), lower the image intensities of urine for the contrast against the wall and have less Partial Volume Effect at the inner border. Shown in FIG. 6(c) is a result of a coupled level set method and PV image segmentation applied to segment the inner and outer borders of the bladder wall from the T₁-weighted MR images.

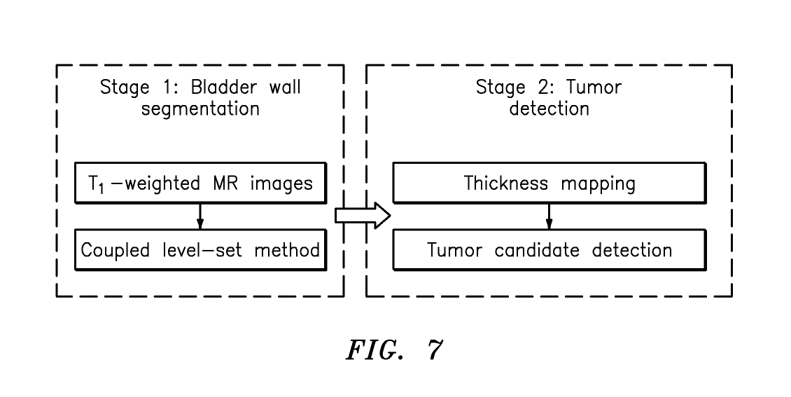

Starting from the segmented bladder wall, the procedure of thickness mapping is conducted on the inner border with a thickness value assigned on each voxel of the inner border. Bladder tumors with various sizes bulge into the lumen area from the inner border in various shapes, like polypoid, sessile, abnormal plaques, and even flat. However, they share a common feature of being protrusions out of the bladder wall, which leads to a sudden change of bladder wall thickness. Such an abnormality can be detected through using of a blob detector on the two-dimension flattened inner border. FIG. 7 is a flowchart of a method of an embodiment of the present invention.

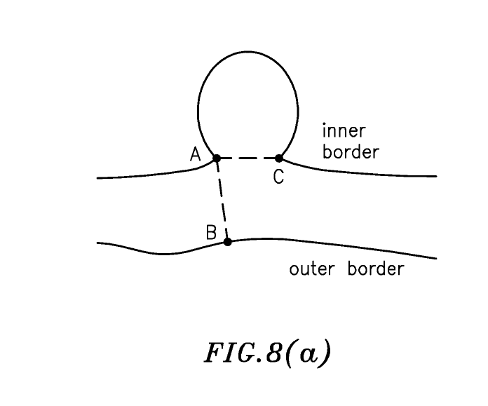

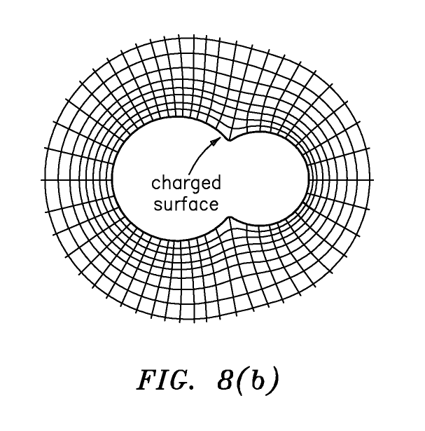

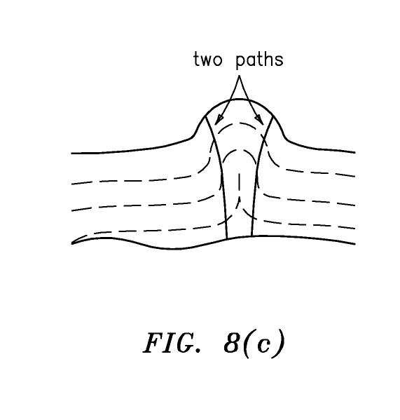

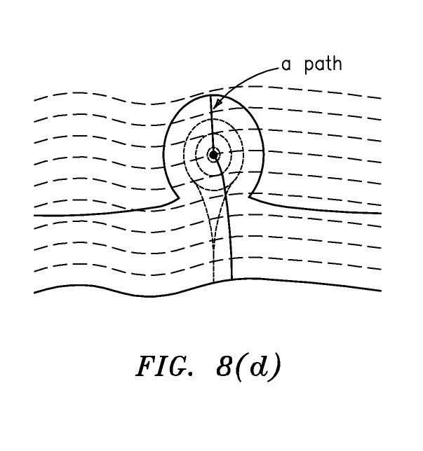

The segmented inner and outer borders are spatial three-dimensional surfaces, with ‘thickness’ used to mean a length of a path starting from a point on one surface and ending at another point on the other surface, and the path is constrained by a local shape of the two surfaces. As shown in FIG. 8(a), the desired path starting from point ‘A’ would be the dashed line ‘AB’ instead of ‘AC’. In the present invention, the two borders are assumed as two iso-potential surfaces which generate electric potential between them, and the integral path is traced along the gradient direction of the potential field, as shown in FIG. 8(b). An exact implementation of the idea in continuous space is rather complicated and, therefore, is simplified based on the voxel units. In this embodiment of the present invention, a potential field located inside the wall is explored via a CAD scheme for bladder tumor detection based on the resulted thickness mapping.

Accurate computation of the electric potential between the two surfaces would otherwise be rather complicated and time consuming. Distance Transform (DT) based on the inner/outer border has similar properties as that of electric potential field. The iso-distance surfaces are smooth and not self-intersecting and there is only one path if tracing is performed along the gradient direction of the DT. As shown in FIG. 8(b), the closed thin curves are also assumed as the iso-distance surfaces of the DT based on the charged surface. Therefore, the DT is utilized to approximate the electric potential field. A fast marching method is used to determine the DT inside the bladder wall. The dotted, i.e. near horizontal, curves in FIGS. 8(c) and 8(d) represent the iso-distance surfaces based on the top thick solid curve. In the method of this embodiment, starting from a point on the inner border, tracing is performed along the gradient direction of the DT based on the inner border towards the outer border, and the tracing stops upon reaching the outer border.

As shown in FIG. 8(c), the two solid, i.e. near vertical, curves between the two borders are two paths traced along the gradient direction of the DT based on the inner border. As shown in FIG. 8(d), the near vertical solid curve (toward the top of the suspected lesion) representing a path is traced from the inner border towards the outer border. However, the tracing will stop at the center of the lesion since the DT converges there, and such convergence actually indicates the abnormality. The tracing process is further continued to reach the outer border by following the reverse direction of the gradient of the DT based on the outer border. As shown in FIG. 8(d), the (near vertical) solid curve (from the lesion center toward the bottom solid line) denotes the part of the path generated by the second tracing.

Utilizing the method described above, tumor detection is performed via two-dimensional gray images, wherein abnormalities appear as isolated brighter patches or blobs than their surroundings. Such abnormalities can straightforwardly be detected with a two-dimensional blob detector based on the Laplacian of the Gaussian (LoG), as in Equation (1):

∇²L=Lₓₓ+L_{yy} (1)

where Lₓₓ and L_{yy} are the second order derivatives of the convolved image by a Gaussian kernel, as in Equation (2):

L(x, y)=g(x, y, σ)×I(x, y) (2)

where g is the Gaussian kernel with scale σ, and I is the flattened 2D image with the texture of thickness mapping. With this method, the LoG gives strong positive responses for dark blobs and strong negative response for bright blobs. The interest is in the bright blobs. The scale σ is set to be 3 mm so as to focus on lesions larger than 3 mm. Heuristic threshold is applied to the two cases so that pixels with smaller LoG response are labeled and clustered to form the final detections.

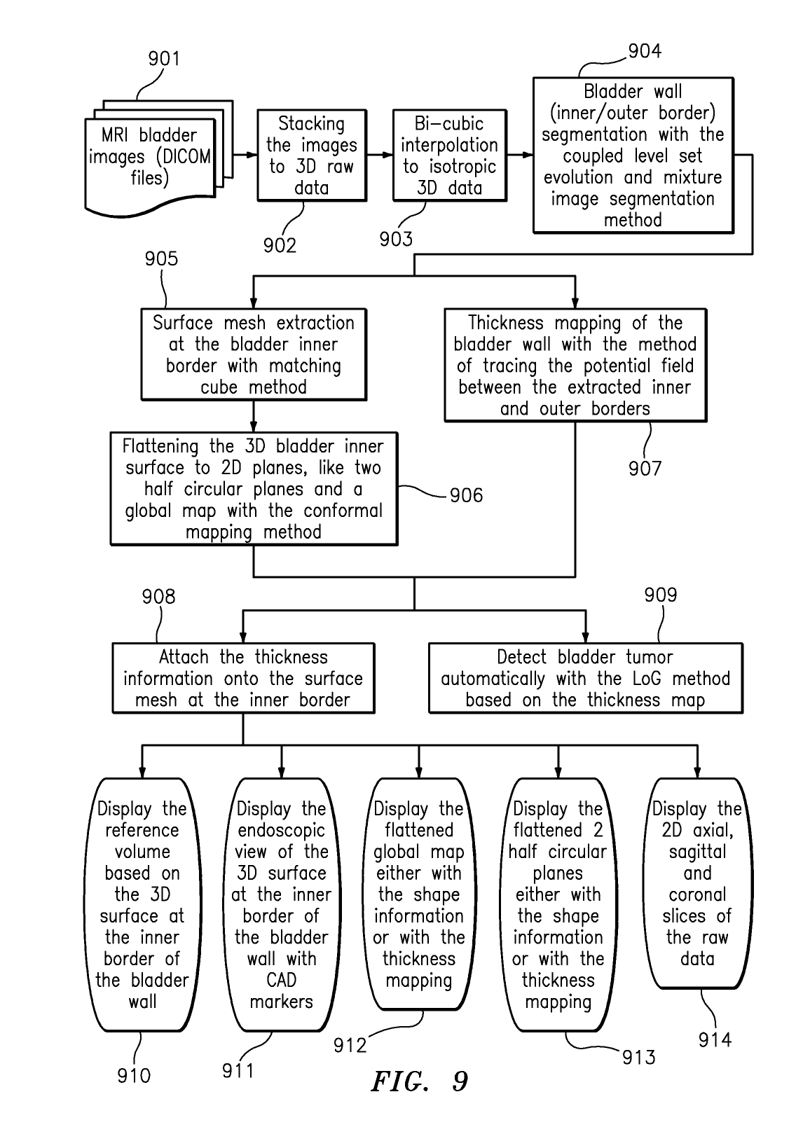

In the present invention, a plurality of MRI bladder images is obtained in step 901 in FIG. 9. In step 902 the images are stacked as three-dimensional raw data and at step 903 bicubic interpolation is performed to obtain isotropic image voxel dimension in the data. At step 904 bladder wall segmentation is performed, followed by a surface mesh extraction and flattening, in steps 905 and 906, and a thickness mapping of the bladder wall is preferably simultaneously performed, in step 907. The mapped thickness distribution is then integrated into the surface mesh for display or visualization in step 908, while the mapped thickness distribution is analyzed for feature selection toward CAD in step 909. The results of the above steps are displayed with steps 910-914 in the corresponding windows in the interface, as shown in FIG. 9.

While the invention has been shown and described with reference to certain exemplary embodiments of the present invention thereof, it will be understood by those skilled in the art that various changes in from and details may be made therein without departing from the spirit and scope of the present invention as defined by the appended claims and equivalent thereof.

What is claimed is:

1. A method for detection of a bladder wall tumor, the method comprising:
creating, by magnetic resonance imaging, a plurality of layers of a bladder wall, wherein a group of voxels having a lowest intensity is identified in one layer of the plurality of layers from a T₁-weighted MRI image;
utilizing an energy function modification to enlarge the one layer of the bladder wall;
utilizing a partial volume image segmentation to obtain tissue type mixture percentages in each voxel near an inner border and an outer border of the bladder wall in the one layer of the bladder wall and obtaining a bladder wall thickness;
obtaining a range of uncertainty at the inner border and at the outer border of the bladder wall; and
integrating, over the range of uncertainty, the bladder wall thickness along a path starting at a point on the outer border of the bladder and ending at a corresponding point on the inner border of the bladder, wherein the path is constrained by a local shape between the two points.

2. The method of claim 1, wherein the identifying of the group of voxels having a lowest intensity is obtained using a T₂-weighted image of the bladder.

3. The method of claim 1, wherein the path is a path of thickness measure that mimics an electric path between two iso-potential surfaces on the inner border and the outer border of the bladder wall.

4. The method of claim 1, wherein the obtained tissue type mixture percentages indicates a tissue type of one of urine, bladder wall, and a fat/muscle mixture.

5. The method of claim 1, wherein a voxel having a tissue type mixture percentage of less than five percent is ignored and remaining voxels are determined to represent the bladder wall.

6. The method of claim 2, wherein the integration is a summation of tissue type mixtures in the voxels along the path of thickness measure.

7. The method of claim 1, wherein the inner border of an irregular three-dimensional bladder is deformed by a conformal mapping into a sphere.

8. The method of claim 7, wherein the deformed sphere is flattened by the conformal mapping into two disks.

9. The method of claim 8, wherein the two disks are divided into regions of interest.
